(19) United States
(12) Patent Application Publication
(10) Pub. No.: US 2009/0066341 A1
(43) Pub. Date: Mar. 12, 2009

(54) MEDICAL DEVICE AND TEST METHOD FOR MEDICAL DEVICE

(75) Inventor: Takehiro YOSHIMURA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/206,160

(22) Filed: Sep. 8, 2008

(30) Foreign Application Priority Data
Sep. 12, 2007 (JP) 2007-237048

(51) Int. Cl. G01R 31/02; H05K 5/00; A61B 8/00

(52) U.S. Cl. 324/537; 361/752; 600/437

(57) ABSTRACT

A medical device of the present invention is a medical device having a plurality of wiring boards disposed in a housing, each of which has a board terminal for testing the wiring board, comprising: one or more external terminals provided on the outer surface of the housing and connected to one or more of the board terminals; and one or more block portions provided between the respective board terminals, and the respective external terminals and blocking writing of data to the wiring boards.

### CROSS REFERENCE TO RELATED APPLICATION

This application claims the benefit of Japanese Application No. 2007-237048 filed in Japan on Sep. 12, 2007, the contents of which are herein by this reference.

### BACKGROUND OF THE INVENTION

1. Field of the Invention

The present invention relates to a medical device and a test method for a medical device.

2. Description of the Related Art

Recently, as medical devices have become sophisticated and complex, it is getting not easy, when a medical device has a malfunction, to inspect and identify the location of the malfunction.

Therefore, a technique has been proposed: performing maintenance work or the like of a medical device from a remote location using communication lines in order to prevent a malfunction in advance.

For example, Japanese Patent Application Laid-Open Publication No. 2001-327497 discloses a remote maintenance method for a medical image diagnostic device, in which change of system setting of a medical image diagnostic device is performed by a computer at a remote location, connected through a network.

In addition, Japanese Patent Application Laid-Open Publication No. 2005-058574 discloses an ultrasound diagnostic device in which a service person can reliably and readily perform maintenance work of the main body side of the ultrasound diagnostic device on the remote side without going to the main body side of the ultrasound diagnostic device.

In the case of an ordinary device which is not a medical device, when a malfunction of the device occurs, a repairer can perform close inspection after disassembling the device having a malfunction on the site to find out the location of the malfunction.

However, specified medical devices such as an ultrasound diagnostic device must not only be manufactured or sold without permission by relevant authorities according to the law (such as Pharmaceutical Law), but must not be disassembled except in a place of business having a license for repairing business, equipped with buildings and facilities specified by Regulations for Buildings and Facilities for Pharmacies and the like.

FIG. 8 is a diagram of the configuration of a conventional ultrasound diagnostic device 1d, and FIG. 9 is a diagram of the configuration at the time of testing the conventional ultrasound diagnostic device 1d.

As shown in FIG. 9, in order to test wiring boards 11, 12, 13 disposed in a housing 3a of the device of the ultrasound diagnostic device 1d, an outer surface panel 3A covering the housing 3a needs to be removed to connect board terminals 21, 22 or 23 and test portion 100 through an opening portion 3B.

### SUMMARY OF THE INVENTION

An ultrasound diagnostic device which is a medical device of the present invention is a medical device having a plurality of wiring boards disposed in a housing of a device, each of which has a board terminal for testing the wiring board, comprising: one or more external terminals provided on an outer surface of the housing of the device and connected to one or more of the board terminals; and one or more block portions provided between the board terminal and the external terminal for blocking writing of data to the wiring boards.

Additionally, a test method for an ultrasound diagnostic device which is a medical device of the present invention is a test method for a medical device having a plurality of wiring boards disposed in the housing of a device, each of which has a board terminal for testing the wiring board, comprising one or more external terminals provided on the outer surface of the housing of the device and connected to one or more of the board terminals, wherein: test portion is connected to one or more external terminals; one or more block portions for blocking writing of data to the wiring boards is disposed between the board terminal and the test portion; and the test portion performs testing of the wiring boards through the external terminal while writing of data to the wiring boards is blocked.

### BRIEF DESCRIPTION OF THE DRAWINGS

FIG. 1 is a diagram of a configuration of an ultrasound diagnostic device of a first embodiment of the present invention;

FIG. 2 is a schematic function block diagram of data writing block portion of the first embodiment of the present invention;

FIG. 3 is a diagram showing an example of connection of a wiring board having a board terminal of the JTAG standard;

FIG. 4 is a flow chart for performing testing of a wiring board using the board terminal of the JTAG standard;

FIG. 5 is a diagram of a configuration for describing a test method for a medical device of the first embodiment;

FIG. 6 is a diagram of a configuration of a medical device of a second embodiment of the present invention;

FIG. 7 is a diagram of a configuration showing a test method for a medical device of a third embodiment of the present invention;

FIG. 8 is a diagram of a configuration of a conventional ultrasound diagnostic device; and

FIG. 9 is a diagram of a configuration at the time of performing testing of a conventional ultrasound diagnostic device.

### DETAILED DESCRIPTION OF THE PREFERRED EMBODIMENTS

Embodiments of the present invention will be described below with reference to the drawings.

### First Embodiment

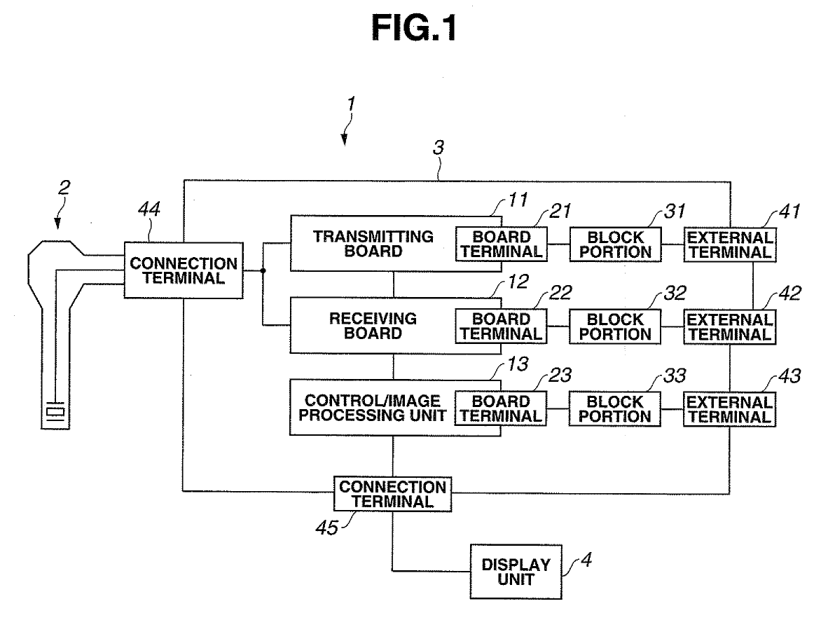

FIG. 1 is a diagram of a configuration of an ultrasound diagnostic device of a first embodiment of the present invention.

As shown in FIG. 1, an ultrasound diagnostic device 1 which is a medical device of the present embodiment has a housing of the device (hereinafter referred to as “housing”) 3, an ultrasound endoscope 2 and a display unit 4. The housing 3 has a connection terminal 44 to which the ultrasound endoscope 2 is attachable and detachable and a connection terminal 45 to which the display unit 4 is attachable and detachable. In addition, the housing 3 has three external terminals 41, 42, 43 on the outer surface of the housing.

The housing 3 is a box covered by outer walls, accommodating control portion and the like of the ultrasound diagnostic device: a box which must not be disassembled except in a place of business having a license for repairing business, equipped with buildings and facilities specified by Regulations for Buildings and Facilities for Pharmacies and the like according to the law (such as Pharmaceutical Law), as described above. That is, the housing 3 refers to members constituting the outer walls, except members removal of which causes no problem from a legal standpoint such as a readily removable cover for preventing contamination and a readily removable panel for preventing erroneous operation.

In the housing 3, a plurality of wiring boards are disposed on which various semiconductor components and the like are mounted. As examples of major wiring boards, FIG. 1 shows three wiring boards: a wiring board 11 for transmitting processing, a wiring board 12 for receiving processing and a wiring board 13 for control/image processing. Each of the above wiring boards has FPGA (Field Programmable Gate Array) function and provides each function by programming.

The wiring boards 11, 12, 13 of the ultrasound diagnostic device 1, which is a medical device of the present embodiment, are provided with board terminals 21, 22, 23 for testing the various semiconductor components mounted on the wiring boards, respectively. These board terminals are board terminals which have come to be provided since it has become difficult to connect a test probe to each of the terminals to perform testing because the semiconductor components have come to have many terminals in order to adapt to high-density mounting; and are hereinafter referred to as board terminals.

That is, in FIG. 1, each of the wiring boards 11, 12, 13 has a board terminal and each board terminal is connected to an external terminal. Specifically, the board terminals 21, 22, 23 are respectively connected to the external terminals 41, 42, 43. However, not all the wiring boards in the housing 3 need to have a board terminal. In addition, there may be a wiring board having a board terminal but being not connected to an external terminal. That is, only board terminals which need to be tested without disassembling the housing 3 may particularly be connected to external terminals, considering the frequency of occurrence of malfunctions of each wiring board or the like.

To be exact, as shown in FIG. 1, the external terminals 41, 42, 43 are provided between the inner surface and the outer surface of the housing 3, and are connected to the board terminals 21, 22, 23 through block portions 31, 32, 33 in the inner surface of the housing 3.

Each of the three board terminals in the housing 3 of the present embodiment is connected to an external terminal through the block portion. The data writing block portions 31, 32, 33 are provided between the board terminals 21, 22, 23 on the three wiring boards and the external terminals 41, 42, 43 on the outer surface of the housing 3, corresponding to the respective board terminals and the respective external terminals.

In the following, each of the board terminals 21, 22, 23 is referred to as a board terminal 20, each of the block portions 31, 32, 33 is referred to as block portion 30, and each of the external terminals 41, 42, 43 is referred to as an external terminal 40. Each of the board terminals 20 and each of the external terminals 40 represent a terminal group respectively having a plurality of physical terminals for connecting a plurality of wires. A board terminal of the JTAG standard described later is, for example, a terminal group comprising four wires and one option wire: a total of five wire terminals.

The block portion 30 is provided for each board terminal 20 because the board terminal 20 for testing the wiring board not only can test the wiring board, that is, test the semiconductor components (such as CPU, FPGA and EPROM) mounted on the wiring board, but can also rewrite or newly write the data such as programs and parameters recorded in the semiconductor components to be tested, through the board terminal 20.

In the case of an ordinary device other than medical devices, function such as rewriting of data is useful function for easily performing version upgrade work of the software. However, such function of the board terminal causes a problem in testing the wiring boards of a medical device as described above.

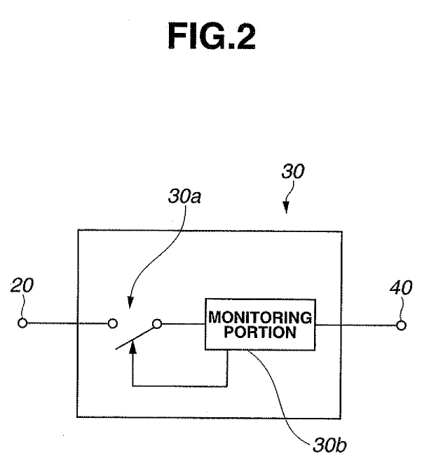

FIG. 2 shows a schematic function block diagram of the data writing block portion 30 of the present embodiment. The block portion 30 is provided between the board terminal 20 and the external terminal 40. The block portion 30 comprises a switch portion 30a and a monitoring portion 30b. The monitoring portion 30b always monitors whether or not there is a signal to write data to the semiconductor components mounted on the wiring board. Operation of writing data to the semiconductor components on the wiring board is, from the standpoint of the semiconductor components, reading of data.

One end of the monitoring portion 30b is connected to the external terminal 40 and the other end is connected to the board terminal 20 through the switch portion 30a. The switch portion 30a is switched ON or OFF depending on a scan signal from the monitoring portion 30b. For example, the switch portion 30a is usually ON: the signal from the external terminal 40 being supplied to the board terminal 20 and also the signal from the board terminal 20 being supplied to the external terminal 40. But when the signal from the external terminal 40 is a signal to write data to a memory of a semiconductor component in the wiring board, the monitoring portion 30b detects that writing signal and outputs a control signal for the switch portion 30a to be OFF.

Specifically, the block portion 30 always monitors a signal to start writing of data to the wiring board, and stores in a predetermined memory area in the block portion 30 flag data in which the state of no signal to start writing of data is 0, whereas the state of detecting a signal to start writing of data is 1. The block portion 30 switches the switch portion 30a ON, that is, providing connection between the external terminal 40 and the board terminal 20 when the flag is 0, but switches the switch portion 30a OFF, that is, providing release between the external terminal 40 and the board terminal 20 to block writing of data when the flag is 1.

Here, the signal to start writing of data to the wiring board is a signal transmitted at the beginning of the start of operation of writing data from the outside to a memory in a semiconductor component mounted on the wiring board. The type of data of the signal to start writing of data differs depending on the kinds of the semiconductor components, the dedicated applications for testing the wiring board or the like. Therefore, the block portion detects the signal to start writing of data corresponding to the medical device to be tested.

From the viewpoint of the fail-safe at the time of a malfunction of the block portion, preferably the block portion 30 usually keeps the switch portion 30a OFF, and provides connection only when the monitoring portion 30b can confirm that there is no signal to start writing.

The ultrasound diagnostic device 1 which is a medical device of the present embodiment has the block portion between the board terminal and the external terminal. Therefore, testing of the semiconductor components mounted on the wiring board can be appropriately performed using the board terminal in a medical device whose modification of the software is not permitted.

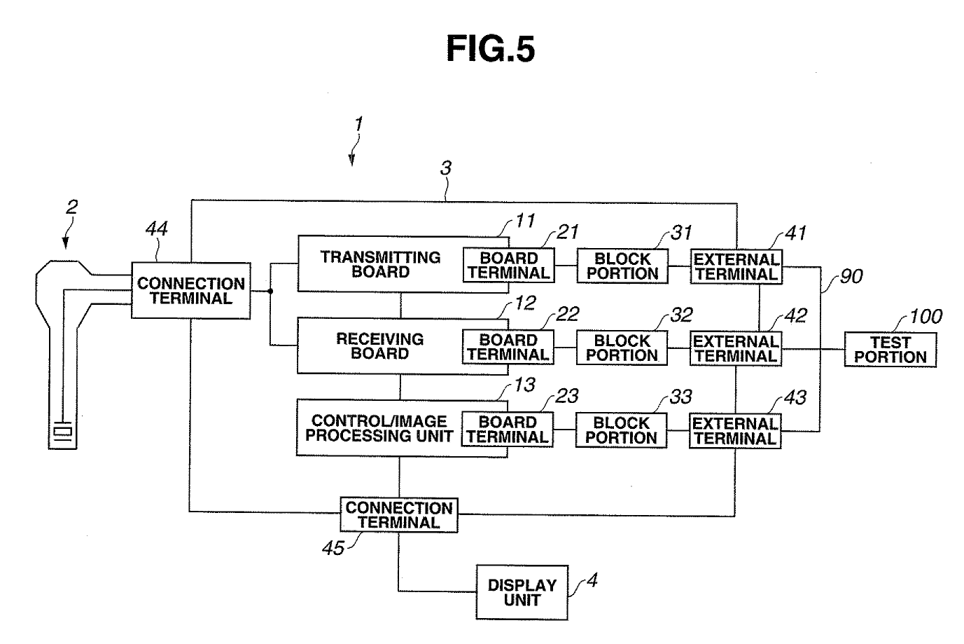

For the board terminals 21, 22, 23, the check board terminal of the JTAG standard is preferable. In the following, an example of a medical device will be shown in which the check board terminal of the JTAG standard is used as the board terminal for testing the wiring board. FIG. 5 is a diagram of a configuration for describing a test method for the ultrasound diagnostic device 1 which is a medical device of the first embodiment.

The JTAG standard was proposed by Joint European Test Action Group (JETAG: now JTAG) in 1985, and was established as a standard by Institute of Electrical and Electronics Engineers (IEEE) in 1990 as IEEE std. 1149.1-1990 “Standard Test Access Port and Boundary-Scan Architecture”, setting forth a standard of the “boundary scan test” (boundary scan examination). The JTAG standard is a standard established for the purpose of solving problems arising from the difficulty in connecting probes physically to semiconductor components on which the BGA (Ball Grid Array) is mounted or to multilayer printed boards having many input/output pins, and performing testing.

A serial bus of the JTAG standard provides portion for access from the test portion for performing a boundary scan on each of the semiconductor components mounted on a wiring board. Therefore, the test portion can test the inactive semiconductor components. By the test, the test portion can verify the connection of the semiconductor components and can verify that they are properly attached and interconnected, that is, can test the wiring board.

A serial bus of the JTAG standard can interconnect one or more semiconductor components in the form of a chain, and the test portion can designate any of the semiconductor components by addresses. Since usually a plurality of devices of the wiring board are interconnected to the JTAG bus, the serial bus of the JTAG standard is sometimes called a STAG chain.

The serial bus of the JTAG standard uses four lines and one option line. These lines include a serial data input line, a serial data output line, a clock line and a test mode select line. Usually, the data output line of the first semiconductor component of the chain is connected to the data input line of the second semiconductor component of the chain, and the data output line of the second semiconductor component is connected to the data input line of the third semiconductor component. That is, the data input lines and the data output lines of a plurality of semiconductor components are connected in a daisy chain configuration.

IEEE1152 is an extended new version of 1149.1JSTAG. In the present invention, the JTAG includes both variations of 1149.1 and 1152, and also includes versions having further developed similar function.

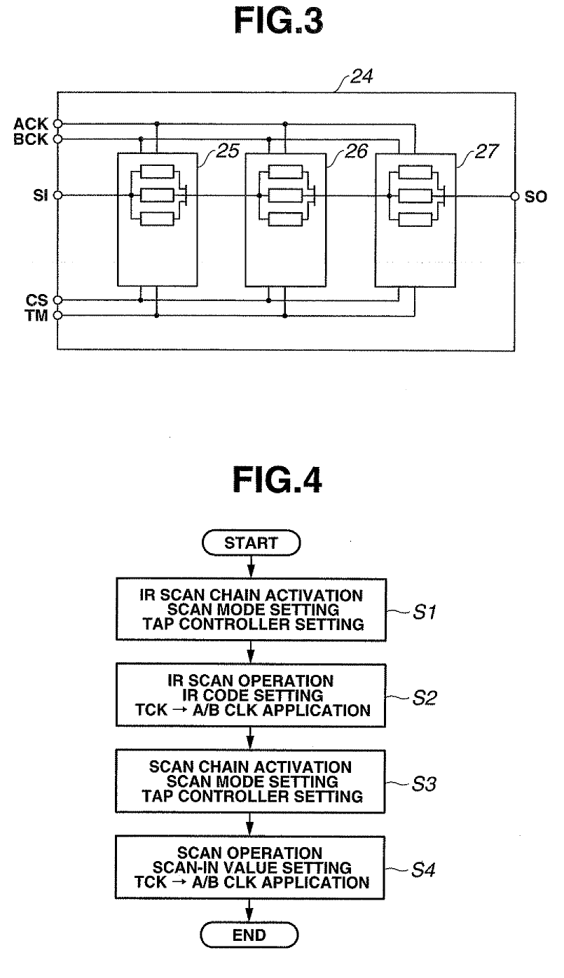

FIG. 3 is a diagram showing an example of connection of a wiring board 24 having a board terminal of the JTAG standard. In FIG. 3, LSI 25, 26 and 27, which are semiconductor components mounted on the wiring board 24, have provided therein control signal generating portion including an unshown instruction register and scan A/B clock generating portion which generates clocks of two phases of scan A/B phases. The state of the control portion in the semiconductor components LSI 25, 26 and 27 is set by a sequence signal of the connected test portion to perform a scan.

In FIG. 3, ACK is a terminal for the A clock signal and BCK is a terminal for the B clock signal. A scan board terminal SI is a terminal for inputting a scan signal and a scan output terminal SO is a terminal for outputting the scan signal. A chain select CS is a chain select terminal for selecting a range connected in the same scan chain, and a test mode select signal board terminal TM is a terminal for a signal which selects a test mode. The above terminal group corresponds to the board terminal 20.

FIG. 4 shows a flow chart for testing the wiring board 24 using the board terminal of the JTAG standard. Specifically, in the flow chart shown in FIG. 4, in order to activate the chain connection of the instruction register scan system in which instruction registers (referred to as IR register) for setting instructions for performing testing are serially connected, first the scan mode is set and a TAP controller, which is JTAG test system control portion, is set (S1). After the scan mode has been set and the TAP controller has been set, in order to perform scan operation of the instruction register, instruction register code is set and input voltage is applied from the test clock terminal to the scan A/B phase clock terminal (S2).

After the setting of the instruction register code and the voltage application from the test clock terminal to the scan A/B clock terminal, in order to activate the chain connection of the scan, the scan mode is set and the TAP controller is set (S3). After the scan mode has been set and the TAP controller has been set, in order to perform scan operation, a scan-in value to be inputted to the scan board terminal is set and input voltage is applied from the test clock terminal to the scan A/B clock terminal (S4). Thus a signal for setting the scan-in value to be inputted to the scan board terminal is provided and input voltage is applied from the test clock terminal to the scan A/B clock terminal, ending the test processing. The above test is automatically performed in accordance with content set in advance upon activation of a JTAG application which is application software for the STAG test and which the test portion 100 has.

As shown in FIG. 5, the three wiring boards 11, 12, 13 disposed in the housing 3 of the ultrasound diagnostic device 1 respectively have the board terminals 21, 22, 23 of the JTAG standard. The board terminals 21, 22, 23 of the JTAG standard on the wiring boards 11, 12, 13 are respectively connected through the block portions 31, 32, 33 to the external terminals 41, 42, 43 provided on the outer surface of the housing 3. Here, the outer surface of the housing 3 of the device is the outer wall surface of the device.

First, the test portion 100 equipped with the JTAG application is connected to the external terminals 41, 42, 43 through a cable 90. Although the test portion 100 is connected to the three external terminals 41, 42, 43 in FIG. 5, it may be connected only to one or two external terminals to which the board terminal of the wiring board to be tested is connected.

Since the ultrasound diagnostic device 1 which is a medical device of the present embodiment is a medical device, the housing of the device cannot be disassembled on the site. However, since the outer surface of the housing 3 has the external terminals, the test portion 100 can be connected to the board terminal of each wiring board through the external terminal.

Specifically, in the medical device of the present embodiment, the test portion 100 equipped with a JTAG interface and a check application, for example a PC, can be connected to the external terminal 40 for the JTAG check on the outer surface of the housing 3. When a general-purpose PC not containing a JTAG interface is used as the test portion 100, connection is made with a JTAG interface being interposed between the external terminal 40 for the JTAG check and the PC.

Next, the JTAG application mounted on the test portion 100 is activated, an initial setting of test content adapted to the medical device to be tested is made and an actual test is performed.

Therefore, condition of the wiring boards in the device can be easily tested from the outside to diagnose a malfunction without disassembling the housing 3 on the site. In addition, when it is found that the cause of the malfunction exists in a particular wiring board accommodated in the housing 3, the main body of the device accommodating that wiring board is taken to a factory permitted to repair medical devices to perform disassembly and exchange work. When the device is taken to the factory, the location of the malfunction has been found in advance so that the time for disassembly work can be minimized, thereby improving the electrical safety of repair work. In addition, reduction of cost for maintenance and tests of the medical device becomes possible.

Further, since the wiring board having a malfunction is found, if that malfunctioning part is not a part of essential function of the medical device, often other function can be used without problems as long as the function in question is not used. For example, if it is found that the wiring board for controlling color Doppler function is the cause of the malfunction in an ultrasound diagnostic device, it is found that normal endoscope function and B-mode ultrasound diagnosis can be used safely. Therefore it is possible to get out of the worst situation of the medical device being wholly unusable.

In the ultrasound diagnostic device 1 which is a medical device of the present embodiment, the test portion 100 is connected to the board terminal 20 of the JTAG standard without disassembling the ultrasound diagnostic device 1 to perform a test so that whether or not the wiring board has a malfunction can be tested appropriately and quickly without risk of modification of data.

### Second Embodiment

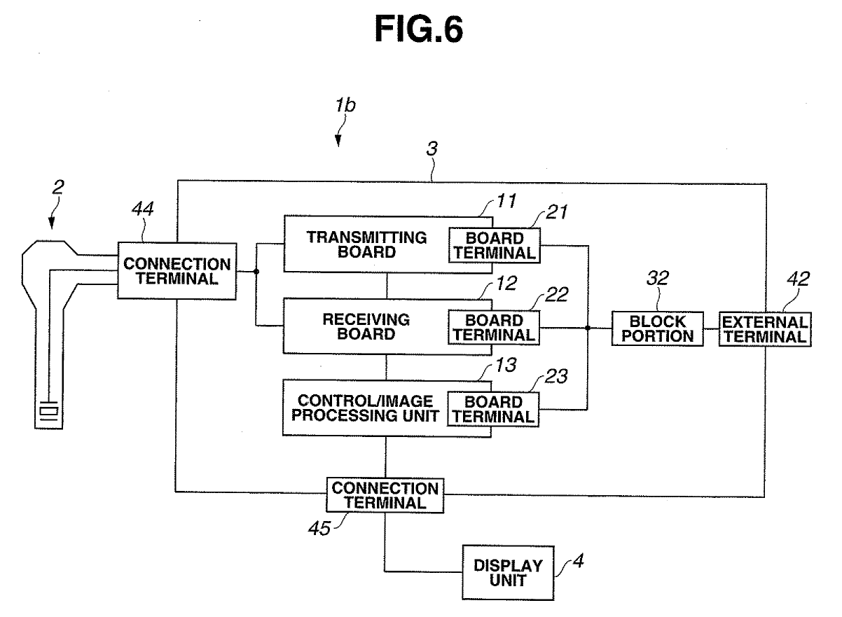

FIG. 6 is a diagram of the configuration of a medical device 1b of a second embodiment of the present invention.

The difference from the first embodiment is that three board terminals 21, 22, 23 on three wiring boards are connected to one block portion 32 which is then connected to one external terminal 42 on the outer surface of a housing 3.

A complicated medical device uses a large number of wiring boards and therefore has a large number of the external terminals 40 provided on the outer surface of the housing 3. In addition, if the block portion 30 is provided for each of the board terminals 20 as shown in the first embodiment, a large number of block portion 30 are needed.

For this reason, in the medical device of the second embodiment, the board terminals 21, 22, 23 for the JTAG check on the wiring boards are connected to one block portion 32, and the one block portion 32 is connected to one external terminal 42 to achieve the same object as the first embodiment.

### Third Embodiment

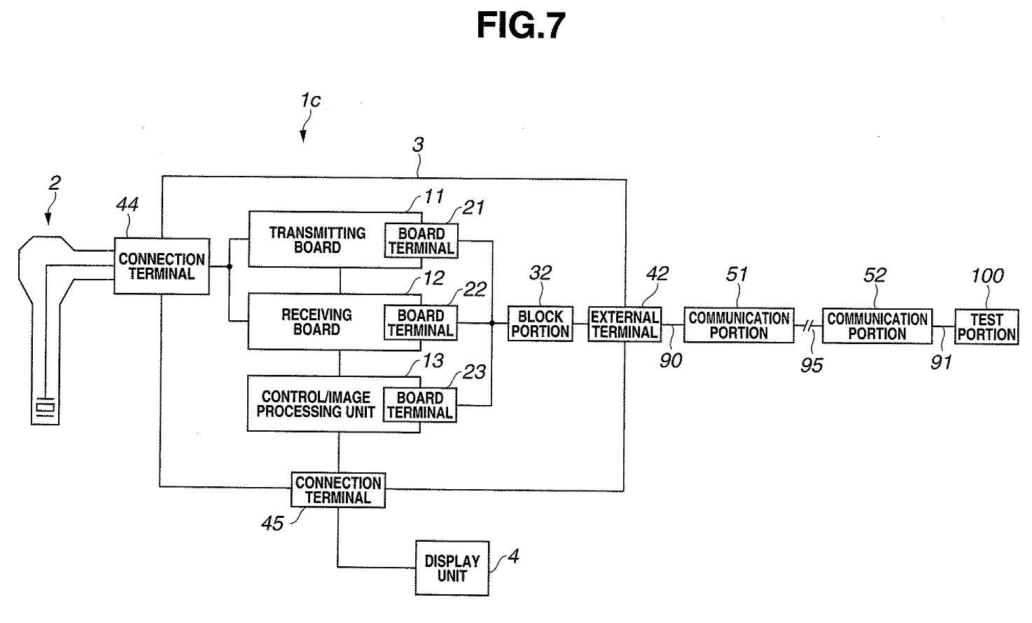

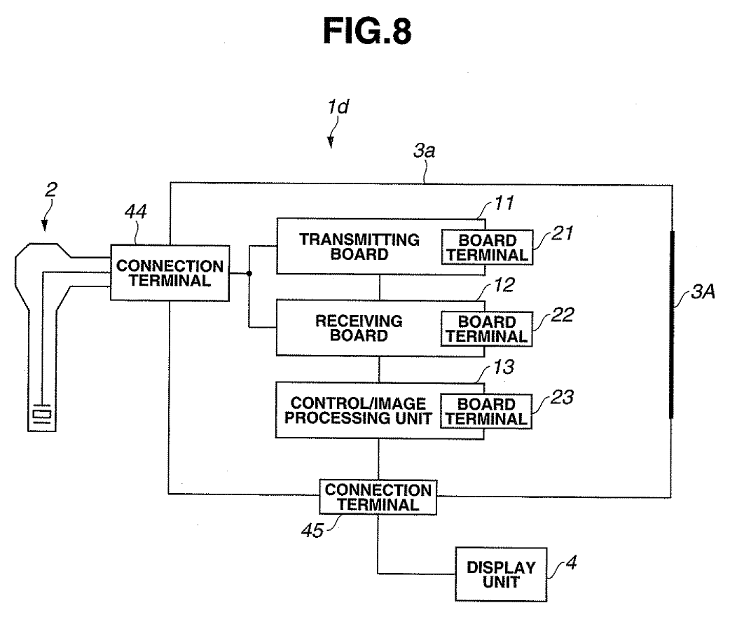

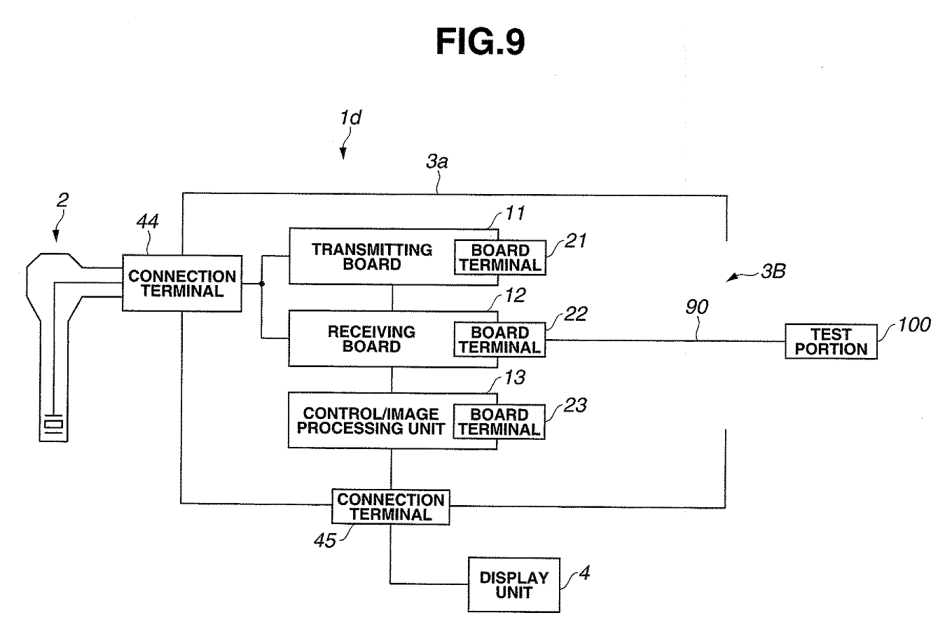

FIG. 7 is a diagram of a configuration showing a test method for a medical device 1c of a third embodiment of the present invention. In the present embodiment, communication portion 51 having communication function is connected to an external terminal 42 on the outer surface of a housing 3, and is connected through a communication line 95 via communication portion 52 to the test portion 100.

By connecting the test portion 100 to the external terminal 42 through the communication portion 51 and 52, the medical device 1c can be tested from a remote location. For the communication line 95, LAN, the Internet or a cellular phone line is preferably used.

In the present embodiment, the block portion 32 only needs to be disposed between the board terminal 20 and the test portion 100. That is, the block portion 32 does not need to be disposed in the housing 3.

The test method for the medical device of the present embodiment is a test method for a medical device having: a plurality of wiring boards disposed in the housing of the device, each of the wiring boards having a board terminal for testing the wiring board; and one or more external terminals provided on the outer surface of the housing of the device and connected to one or more of the board terminals, wherein: one or more block portions for blocking writing of data to the wiring board is disposed between the board terminal and the test portion; the test portion is connected to one or more external terminals; and data is read from the wiring board by the test portion through each external terminal while writing of data to the wiring board is blocked.

By the test method for the medical device of the present embodiment, a test on the medical device is performed via a communication line, and thereby further cost for maintenance and tests can be reduced as well as a business model for a new service can be built.

Information obtained by operation of medical devices, such as ultrasound diagnostic images, is personal information of a patient and must be strictly managed. Therefore, in the medical device of the present embodiment, a control portion for operational function of the medical device and a portion for control and storage of information obtained by operation of the medical device are preferably independent separate components.

The present invention has been described thus far assuming the current legal system. However, a situation is expected to occur where partial version upgrade work of software of medical devices on site is possible due to an amendment of the law in the future. In such case, data can be written to the semiconductor components and the like mounted on the wiring board temporarily by stopping the function of the block portion 30 of the medical device, or by disposing portion to be connected to the test portion 100 not via the block portion 30 is provided and switching the connected portion.

Having described the preferred embodiments of the invention referring to the accompanying drawings, it should be understood that the present invention is not limited to those precise embodiments and various changes and modification thereof could be made by one skilled in the art without departing from the spirit or scope of the invention as defined in the appended claims.

What is claimed is:

1. A medical device having a plurality of wiring boards disposed in a housing of a device, each of which has a board terminal for testing the wiring board, including:
one or more external terminals provided on an outer surface of the housing of the device and connected to one or more of the board terminals; and
one or more block portions provided between the board terminal and the external terminal and blocking writing of data to the wiring board.

2. The medical device according to claim 1, wherein
the board terminal for testing the wiring board is a board terminal of JTAG standard.

3. The medical device according to claim 1, wherein
the number of the external terminal is one.

4. The medical device according to claim 2, wherein
the number of the external terminal is one.

5. The medical device according to claim 1, wherein
the external terminal can be connected to communication portion.

6. The medical device according to claim 1, wherein
the medical device is an ultrasound diagnostic device.

7. The medical device according to claim 5, wherein
the medical device is an ultrasound diagnostic device.

8. A test method for a medical device having:
a plurality of wiring boards disposed in a housing of a device, each of which has a board terminal for testing the wiring board; and
one or more external terminals provided on an outer surface of the housing of the device and connected to one or more of the board terminals, including:
a test portion connecting step of connecting test portion to the one or more external terminals; and
a test step of testing the wiring board by use of the test portion through the external terminal while blocking writing of data to the wiring board by use of one or more block portions disposed between the board terminal and the test portion and blocking writing of data to the wiring board.

9. The test method for a medical device according to claim 8, wherein
the board terminal for testing the wiring board is a board terminal of JTAG standard.

10. The test method for a medical device according to claim 8, further having:
a communication portion connecting step of connecting communication portion to the external terminal; and
a communication test portion connecting step of connecting the test portion to the external terminal through a communication line.

11. The test method for a medical device according to claim 9, further having:
a communication portion connecting step of connecting communication portion to the external terminal; and
a communication test portion connecting step of connecting the test portion to the external terminal through a communication line.
